(19) United States
(12) Patent Application Publication
(10) Pub. No.: US 2016/0039591 A1
(43) Pub. Date: Feb. 11, 2016

(54) PACKAGING SYSTEMS, DEVICES, METHODS, AND COMPOSITION INCLUDING CANNABINOID UNIT DOSE FORMS

(71) Applicant: Craig E. Kinzer, Seattle, WA (US)

(72) Inventor: Craig E. Kinzer, Seattle, WA (US)

(21) Appl. No.: 14/805,452

(22) Filed: Jul. 21, 2015

Related U.S. Application Data
(60) Provisional application No. 62/027,374, filed on Jul. 22, 2014.

(51) Int. Cl. B65D 75/36; A61K 31/045; A61K 31/015; G06F 19/00; A61K 31/122; A61K 9/00; A61K 31/616; A61K 31/192; A61K 31/352; A61K 31/01

(52) U.S. Cl. CPC B65D 75/36; A61K 31/352; A61K 31/045; A61K 31/015; A61K 31/01; A61K 31/122; A61K 9/006; A61K 31/616; A61K 31/192; G06F 19/3456

(57) ABSTRACT

Systems, devices, methods, and compositions are described for providing, among other things, cannabinoid unit dose forms. Also described are packaging, systems, devices, methods, and compositions including, among other things, phyto-cannabinoid unit dose forms for treating various diseases or disorders.

### CROSS-REFERENCE TO RELATED APPLICATION

This application claims the benefit under 35 U.S.C. §119(e) of U.S. Provisional Patent Application No. 62/027,374 filed Jul. 22, 2014. This provisional application is incorporated herein by reference in its entirety.

### SUMMARY

In an aspect, the present disclosure is directed to, among other things, a unit dose package. In an embodiment, the unit dose package includes at least a first storage location having a first unit dose of at least one cannabinoid and a pharmaceutically acceptable carrier. In an embodiment, the unit dose package includes at least a first storage location having a first unit dose of at least one phyto-cannabinoid and a pharmaceutically acceptable carrier. In an embodiment, the first unit dose comprises from about 0.1 milligram to about 500 milligrams of the at least one phyto-cannabinoid. In an embodiment, the at least one phyto-cannabinoid is tetrahydrocannabinol, or an analogue or derivative thereof.

In an aspect, the present disclosure is directed to, among other things, a unit dose package including a first storage location having a first unit dose of at least one phyto-cannabinoid and a pharmaceutically acceptable carrier. In an embodiment, the unit dose package includes at least a second storage location having a second unit dose of at least one phyto-cannabinoid and a pharmaceutically acceptable carrier. In an embodiment, the second unit dose is different from the first unit dose. For example, in an embodiment, the second unit dose comprises a phyto-cannabinoid amount different from the first unit dose. In an embodiment, the second unit dose comprises a phyto-cannabinoid; terpene ratio different from a phyto-cannabinoid; and/or terpene ratio of the first unit dose.

In an aspect, the present disclosure is directed to, among other things, a sublingual unit dose form including at least one phyto-cannabinoid and a pharmaceutically acceptable carrier. In an embodiment, the sublingual unit dose form includes at least one phyto-cannabinoid, at least one terpene, and a pharmaceutically acceptable carrier.

In an aspect, the present disclosure is directed to, among other things, a method including obtaining user-specific dose-form fabrication information associated with a first unit dose form including at least one phyto-cannabinoid. In an embodiment, the method includes fabricating a plurality of first unit dose forms including at least one phyto-cannabinoid responsive to obtaining the user-specific dose-form fabrication information.

In an aspect, the present disclosure is directed to, among other things, a system including circuitry configured to obtain user-specific dose-form fabrication information associated with a first unit dose form including at least one phyto-cannabinoid. In an embodiment, the system includes circuitry configured to actuate fabrication of a plurality of first unit dose forms including at least one phyto-cannabinoid responsive to obtaining the user-specific dose-form fabrication information.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A and 1B are perspective views of a unit dose package according to an embodiment.

FIG. 2 is a perspective view of a unit dose package according to an embodiment.

FIG. 3 shows a flow diagram of a method according to one embodiment.

FIG. 4 is a schematic diagram of a system according to an embodiment.

### DETAILED DESCRIPTION

Cannabinoid receptors are part of the cannabinoid receptor system in the brain and are involved in a variety of physiological processes including nociception (pain sensation), appetite, lipid metabolism, gastrointestinal motility, cardiovascular modulation, motor activity, mood, and memory. See e.g., Panagiotis et al., The Neuroprotective Role of Endocannabinoids against Chemical-induced Injury and Other Adverse Effects. Journal of Applied Toxicology 33.4: 246-64 Web (2013) (which is incorporated herein by reference). In an embodiment, cannabinoids, cannabidiols, cannabinols, and the like extracted from Cannabis sativa L, may act at peripheral sites and yield analgesia through the action on CB1 and CB2 receptors. See e.g., Jorge et al., J. Pain Res.; 4:11-24. doi: 10.2147/JPR.S9492 (December 2010) (which is incorporated herein by reference). In an embodiment, cannabidiols may have anxiolytic effects both in humans and in animals. See e.g., Bergamaschi et al., Neuropsychopharmacology, 36(6): 1219-1226. doi: 10.1038/npp.2011.6 (May 2011) (which is incorporated herein by reference). In an embodiment, cannabinoids may be effective in treating chemotherapy-induced emesis. See e.g., Williamson et al., Cannabinoids in clinical practice, Drugs, 60(6):1303-14 (December 2000) (which is incorporated herein by reference).

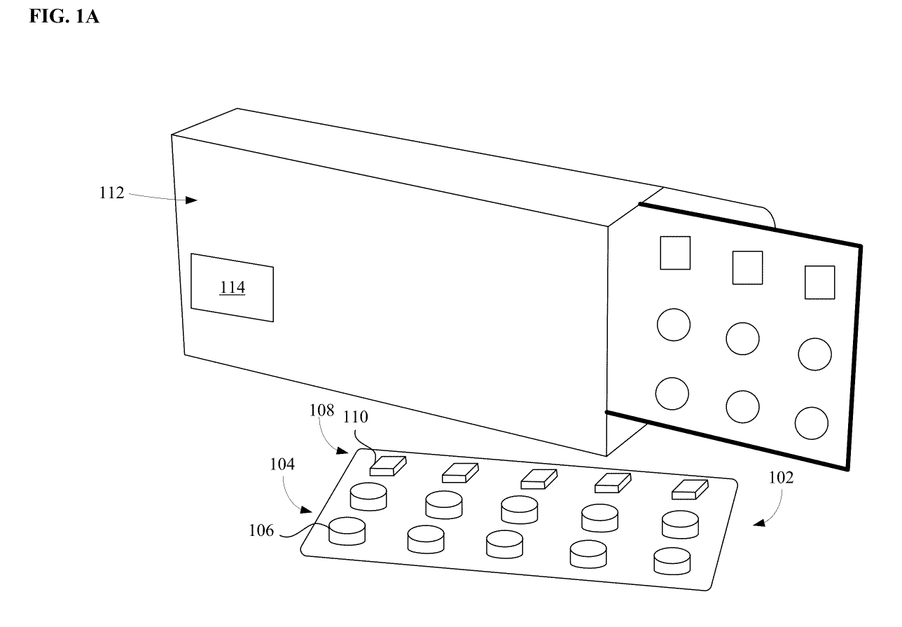

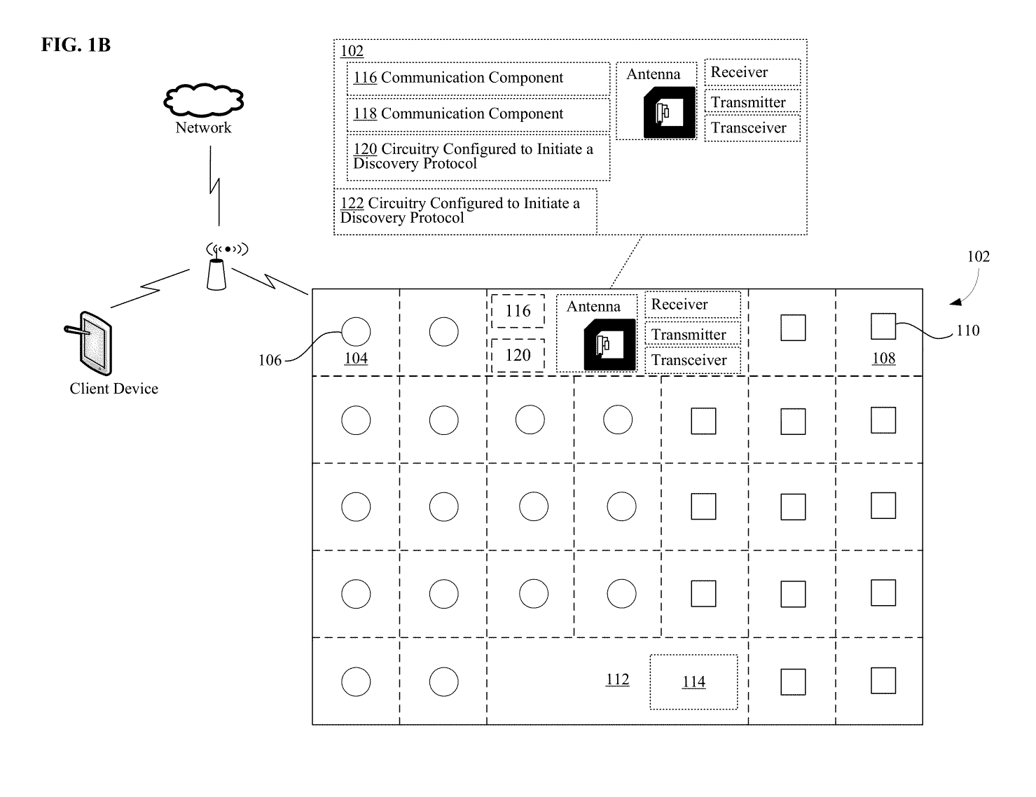

FIG. 1 shows a unit dose package 102 in which one or more methodologies or technologies can be implemented, for example, to treat cannabinoid receptor-mediated diseases or disorders. In an embodiment, one or more of the methodologies or technologies can be implemented to treat cannabinoid receptor-mediated diseases or disorders of the central nervous system (CNS). Diseases or disorders of the central nervous system include, among others, depression, anxiety, attention deficit hyperactivity disorder (ADHD) and the like. Further CNS diseases or disorders include ulcerative colitis; disorders where increased angiogenesis may be beneficial (e.g., diabetes, gangrene, or the like); disorders in which a lack of dopamine or serotonin is involved; disorders in which improved cognition may be beneficial (e.g., Alzheimer's disease, Parkinson's disease, schizophrenia, or the like); Tourette's Syndrome; nausea, vomiting, anorexia nervosa, spasticity, major depressive disorder, cachexia, wasting syndromes, appetite suppression, glaucoma, epilepsy, Dravet Syndrome, multiple sclerosis, asthma, and pain, including pain involved with cancer, HIV, migraines and/or generalized neuropathic pain. In an embodiment, one or more of the disclosed methodologies or technologies can be implemented to treat any disease or disorder that elicits a therapeutic response in a patient using an active agent such as a cannabinoid, or the like.

In an embodiment, the unit dose package 102 includes at least a first storage location 104 having a first unit dose 106 of at least one active agent. In an embodiment, the first unit dose 106 comprises from about 0.1 milligram to about 500 milligrams of at least one active agent. In an embodiment, the first unit dose 106 comprises from about 0.5 milligram to about 250 milligrams of at least one active agent. In an embodiment, the first unit dose 106 comprises from about 1 milligrams to about 100 milligrams of at least one active agent. Non-limiting examples of active agents include cannabinoids cannabidiols, cannabigerols, cannabichromenes, cannabinols, and the like. Non-limiting examples of cannabinoids include those found naturally in cannabis or members of the Cannabis species (e.g., phyto-cannabinoids, phyto-cannabichromenes, phyto-cannabidiols, phyto-cannabidiolic acids, phyto-cannabigerols, phyto-cannabinols, phyto-cannabidivarins, phyto-tetrahydrocannabinolic acids, phyto-tetrahydrocannabivarins, and the like), including Cannabis sativa, Cannabis indica, and Cannabis ruderalis, and chemovars, cultivars, genetic crosses, self-crosses and hybrids thereof. Further non-limiting examples of active agents include synthetic cannabinoids and human cannabinoids (i.e., endocannabinoids), including nabilone, dronabinol, and rimonabant.

Further non-limiting examples of active agents include Δ⁹-tetrahydrocannabinol; Δ⁹-tetrahydrocannabiorcol; Δ⁹-tetrahydrocannabivarin; 10-O-ethylcannabitriol; 6a,7,10a-trihydroxytetrahydrocannabinol; 7,8-dehydro-10-O-ethylcannabitriol; 9,10-epoxycannabitriol; cannabichromene; cannabicitran; cannabicyclol; cannabidiol; cannabidivarin; cannabielsoin; cannabigerol; cannabinol; dihydrocannabinol, and the like, and analogues and derivatives thereof. See e.g., Ross et al., Phytochem Anal, January-February; 16(1), 45-(2005). Further non-limiting examples of active agents include Δ⁹ tetrahydrocannabinol, Δ⁸ tetrahydrocannabinol, cannabidiol, cannabigerol, cannabichromene, cannabinol, and the like, and analogues and derivatives thereof, including ether, ester and amide derivatives. Further non-limiting examples of active agents include phyto-cannabinoids (THC), phyto-cannabichromenes (CBC), phyto-cannabidiols (CBD), phyto-cannabidiolic acids (CBD-A), phyto-cannabigerols (CBG), phyto-cannabinols (CBN), phyto-cannabidivarins (CBDV), phyto-tetrahydrocannabinolic acids (THC-A), phyto-tetrahydrocannabivarins (THCV), and the like.

In an embodiment, the unit dose package 102 includes at least a first storage location 104 having a first unit dose 106 of at least one cannabinoid. For example, in an embodiment, the unit dose package 102 includes at least a first storage location 104 having a first unit dose 106 of at least one phyto-cannabinoid (i.e., plant-derived tetrahydrocannabinols, and the like). In an embodiment, the first storage location 104 includes a first unit dose 106 of at least one phyto-cannabinoid and a pharmaceutically acceptable carrier. In an embodiment, the first unit dose 106 comprises from about 0.1 milligram to about 500 milligrams of the at least one phyto-cannabinoid. In an embodiment, the first unit dose 106 comprises from about 0.5 milligram to about 250 milligrams of the at least one phyto-cannabinoid. In an embodiment, the first unit dose 106 comprises from about 1 milligrams to about 100 milligrams of the at least one phyto-cannabinoid. Other formulations can be used.

In an embodiment, the first unit dose 106 comprises at least about 0.1 milligram of at least one phyto-cannabinoid. In an embodiment, the first unit dose 106 comprises at least about 0.5 milligram of at least one phyto-cannabinoid. In an embodiment, the first unit dose 106 comprises at least about 1 milligram of at least one phyto-cannabinoid. In an embodiment, the first unit dose 106 comprises at least about 1.5 milligrams of at least one phyto-cannabinoid. In an embodiment, the first unit dose 106 comprises at least about 5 milligrams of at least one phyto-cannabinoid. In an embodiment, the first unit dose 106 comprises about 10 milligrams of the at least one phyto-cannabinoid. In an embodiment, the first unit dose 106 comprises about 15 milligrams of the at least one phyto-cannabinoid. In an embodiment, the first unit dose 106 comprises about 20 milligrams of the at least one phyto-cannabinoid. In an embodiment, the first unit dose 106 comprises about 25 milligrams of the at least one phyto-cannabinoid.

In an embodiment, the first unit dose 106 comprises at least one phyto-cannabinoid. In an embodiment, the at least one phyto-cannabinoid is tetrahydrocannabinol or an analogue or derivative thereof. In an embodiment, the at least one phyto-cannabinoid is Δ⁹-tetrahydrocannabinol or an analogue or derivative thereof. In an embodiment, the at least one phyto-cannabinoid is Δ⁸-tetrahydrocannabinol or an analogue or derivative thereof.

In an embodiment, the first unit dose 106 comprises a first terpene. Non-limiting examples of terpenes include borneol, caryophyllene, cineole, delta-3-carene, limonene, D-linalool, β-myrcene, pinene, pulegone, sabinene, terpineol, and the like. In an embodiment, the first unit dose 106 comprises at least one of borneol, β-caryophyllene, cineole, delta-3-carene, limonene, D-linalool, β-myrcene, pinene, pulegone, sabinene, or terpineol. In an embodiment, the first unit dose 106 comprises at least two different terpenes. In an embodiment, the first unit dose 106 comprises at least three different terpenes. In an embodiment, the first unit dose 106 comprises at least four different terpenes.

In an embodiment, the first unit dose 106 comprises a mixture of two or more terpenes. For example, in an embodiment, the first unit dose 106 comprises a mixture of two or more of borneol, β-caryophyllene, cineole, delta-3-carene, limonene, D-linalool, β-myrcene, pinene, pulegone, sabinene, or terpineol. In an embodiment, the first unit dose 106 comprises a mixture of three or more of borneol, β-caryophyllene, cineole, delta-3-carene, limonene, D-linalool, β-myrcene, pinene, pulegone, sabinene, or terpineol. In an embodiment, the first unit dose 106 comprises a mixture of four or more of borneol, β-caryophyllene, cineole, delta-3-carene, limonene, D-linalool, β-myrcene, pinene, pulegone, sabinene, or terpineol.

In an embodiment, the first unit dose 106 comprises one or more flavonoids, flavonoid glycosides (e.g., kaempferol 3-O-sophoroside, quercetin 3-O-sophoroside, etc.), alkanes, esters, or the like. Non-limiting examples of flavonoids include apigenin, quercetin, cannflavin A, β-sitosterol, and the like.

In an embodiment, a first storage location 104 includes a first unit dose 106 of at least one phyto-cannabinoid and a pharmaceutically acceptable carrier. Non-limiting examples of pharmaceutically acceptable carrier materials include binders, disintegrating agents, extenders, fillers, humectants, lubricants, wetting agents, and the like, or mixtures thereof. Further non-limiting examples of pharmaceutically acceptable carrier materials include bentonite clay, calcium stearate, cellulose, cellulose derivatives, cetyl alcohol, citric acid, corn starch, crospovidone, glucose, glycerol monostearate, kaolin, lactose monohydrate, lactose, magnesium stearate, mannitol, microcrystalline cellulose, polyethylene glycols, polysaccharides, polyvinylpolypyrrolidone, silicic acid, sodium chloride, starch, sucrose, talc, and the like, or mixtures thereof.

In an embodiment, the pharmaceutically acceptable carrier comprises a controlled-release carrier. In an embodiment, the pharmaceutically acceptable carrier comprises a controlled-release carrier that exhibits zero order kinetics. Non-limiting examples of controlled-release carrier materials include hydrophilic materials, hydrophilic matrix materials, hydrophobic materials, hydrophobic matrix materials, and the like, or mixtures thereof. Further non-limiting examples of controlled-release carrier materials include polymers, protein derived materials, waxes, shellac, gums, hydrogels, oils, and the like. Further non-limiting examples of controlled-release carrier materials include hydrophilic matrix systems including cellulosic polymers (e.g., hydroxypropylmethylcellulose, methylcellulose, hydroxypropylcellulose, hydroxyethylcellulose, sodium carboxymethylcellulose, and the like).

Further non-limiting examples of pharmaceutically acceptable carrier materials include gum/polysaccharides, polyethylene oxide, homopolymers and copolymer of acrylic acid, and the like. Further non-limiting examples of pharmaceutically acceptable carrier materials include carrageenan, chitosan, crosslinked high amylose starch, guar gum, locust bean gum, pectin, sodium alginate, xanthan gum, and the like. Further non-limiting examples of pharmaceutically acceptable carrier materials include acrylic polymers and copolymers, alkylcelluloses, alkylvinyl polymers, carboxyalkylcelluloses, cellulose ethers, methacrylic acid polymers and copolymers, and the like. Further non-limiting examples of pharmaceutically acceptable carrier materials include beeswax, carnauba wax, fatty acids, fatty alcohols, natural waxes, stearic acid, stearyl alcohol, synthetic waxes, and the like.

In an embodiment, the pharmaceutically acceptable carrier comprises a sustained release carrier. In an embodiment, the pharmaceutically acceptable carrier comprises a sustained release carrier that exhibits first order kinetics. In an embodiment, the pharmaceutically acceptable carrier comprises an immediate release carrier. In an embodiment, the pharmaceutically acceptable carrier comprises an extended release carrier. In an embodiment, the pharmaceutically acceptable carrier includes a disintegrant in an amount sufficient to cause the active agent to exhibit an immediate release profile. For example, in an embodiment, the pharmaceutically acceptable carrier includes a disintegrant in an amount sufficient to cause the at least one phyto-cannabinoid to exhibit an immediate release profile.

In an embodiment, the pharmaceutically acceptable carrier comprises an inhalable composition. In an embodiment, the pharmaceutically acceptable carrier comprises an ingestible composition. In an embodiment, the pharmaceutically acceptable carrier comprises a sublingual composition. In an embodiment, the pharmaceutically acceptable carrier comprises a transdermal composition. In an embodiment, the pharmaceutically acceptable carrier comprises a topical composition. In an embodiment, the pharmaceutically acceptable carrier comprises a transmucosal composition. In an embodiment, the pharmaceutically acceptable carrier comprises a rectal composition.

In an embodiment, the first unit dose 106 comprises a geometric shape indicative of a dose amount. In an embodiment, the shape of the unit dose can be a geometrical shape including regular geometric shapes, such as circular, hexagonal, pentagonal, rectangular, triangular, and the like, as well as irregular geometric shapes. In an embodiment, the number of facets in the shape is indicative of a dose amount. For example, in an embodiment, each facet has a shape is indicative of a dose amount increment (e.g., one facet for every 5 milligrams of active agent). In an embodiment, the first unit dose 106 comprises a geometric shape indicative of a delivery mode.

In an embodiment, the first unit dose 106 comprises a color indicative of a dose amount (e.g., red is indicative of 5 milligrams of active agent, green is indicative of 10 milligrams of active agent, blue is indicative of 15 milligrams of active agent, white is indicative of 25 milligrams of active agent, etc.). In an embodiment, the first unit dose 106 comprises a color indicative of a delivery mode (e.g., red is indicative of inhaled, green is indicative of ingested, blue is indicative of sublingual, white is indicative of transdermal, beige is indicative of topical, purple is indicative of transmucosal, brown is indicative of rectal, etc.).

In an embodiment, the unit dose package 102 includes at least a second storage location 108 having a unit dose of a nonselective COX inhibitor 110 (e.g., aspirin, ibuprofen, naproxen, and the like). In an embodiment, the unit dose package 102 includes at least a second storage location 108 having a unit dose of a nonselective COX inhibitor and a monoamine oxidase inhibitor. Non-limiting examples of monoamine oxidase inhibitors include anthocyanins, curcumin, ginkgo biloba, proanthocyanidin, rhodiola rosea, ruta graveolens, and the like. In an embodiment, the unit dose package 102 includes at least a second storage location 108 having a unit dose of a nonselective COX inhibitor and a nonselective monoamine oxidase inhibitor.

In an embodiment, the unit dose package 102 includes at least a second storage location 108 having a unit dose of at least one nootropic agent. Non-limiting examples of nootropic agents include memory enhancers, neuro enhancers, cognitive enhancers, intelligence enhancers, and the like, or mixtures thereof. Further non-limiting examples of nootropic agents include 8-sulfocholecystokinin octapeptide, acetylcarnitine, ACTH (4-7), ACTH (4-10), adafenoxate, aniracetam, cerebrolysin, choline, cytidine diphosphate choline, donepezil, ergoloid mesylates, etimizol, etiracetam, galantamine, meclofenoxate, nefiracetam, nicergoline, nicotinoyl-GABA, oxiracetam, pantogab, picamilon, piracetam, SA 4503, TA 0910, tacrine, vinpocetine, and the like, or mixtures thereof. See, e.g., “NLM Controlled Vocabulary.” National Center for Biotechnology Information. U.S. National Library of Medicine, n.d. Web. 3 Jul. 2014. In an embodiment, the unit dose package 102 includes at least a second storage location 108 having a unit dose of a nonselective COX inhibitor and at least one nootropic agent.

In an embodiment, the unit dose package 102 comprises a unit-dose blister card. In an embodiment, the unit dose package 102 comprises a portion 112 including regulatory information 114 located thereon.

Referring to FIG. 1B, in an embodiment, the unit dose package 102 includes a communication component 116 that informs a network application regarding contents. In an embodiment, the unit dose package 102 includes a communication component 118 that informs a client device regarding contents. Non-limiting examples of client devices include a wearable device, a smart device, a smart eyewear device, a smart wearable device, a computer device, a laptop computer device, a notebook computer device, a desktop computer device, a cell phone device, a tablet device, a managed node device, a remote controller, an application interface with a smart device, and the like. Further non-limiting examples of client devices include input-output devices, user interfaces, graphical user interfaces, interaction devices, microphones, and the like.

In an embodiment, a component, such as a communication component 116, includes among other things, one or more computing devices such as a processor (e.g., a microprocessor), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, a component includes one or more ASICs having a plurality of predefined logic components. In an embodiment, a component includes one or more FPGAs, each having a plurality of programmable logic components.

In an embodiment, a component includes one or more components operably coupled (e.g., communicatively, electromagnetically, magnetically, ultrasonically, optically, inductively, electrically, capacitively coupled, or the like) to each other. In an embodiment, a component includes one or more remotely located components. In an embodiment, remotely located components are operably coupled, for example, via wireless communication. In an embodiment, remotely located components are operably coupled, for example, via one or more receivers, transmitters, transceivers, antennas, or the like. In an embodiment, the communication component 116 includes a component having one or more routines, data structures, interfaces, and the like.

In an embodiment, a component includes memory that, for example, stores instructions or information. For example, in an embodiment, the communication component 116 includes memory that stores, for example, information regarding user-specific terpene/terpenoid/CBD/THC information, user-specific flavor profile information, user-specific autoimmune diseases information, user-specific pain mitigation profile information, user-specific stress mitigation profile information, user-specific desired feeling/results profile information, and the like. Non-limiting examples of memory include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of memory include Erasable Programmable Read-Only Memory (EPROM), flash memory, or the like. In an embodiment, the memory is coupled to, for example, one or more computing devices by one or more instructions, information, or power buses.

In an embodiment, a component includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, a component includes one or more user input/output components, user interfaces, client devices, or the like, that are operably coupled to at least one computing device configured to control (e.g., electrical, electromechanical, software-implemented, firmware-implemented, or other control, or combinations thereof) at least one parameter associated with, for example, designing, fabricating, formulating, or the like, a unit dose form.

In an embodiment, the unit dose package 102 includes circuitry 120 configured to initiate a discovery protocol that allows the unit dose package and a client device to identify each other and negotiate one or more pre-shared keys. In an embodiment, the unit dose package 102 includes circuitry 122 configured to initiate a discovery protocol that allows the unit dose package and a remote network device to identify each other and negotiate one or more pre-shared keys.

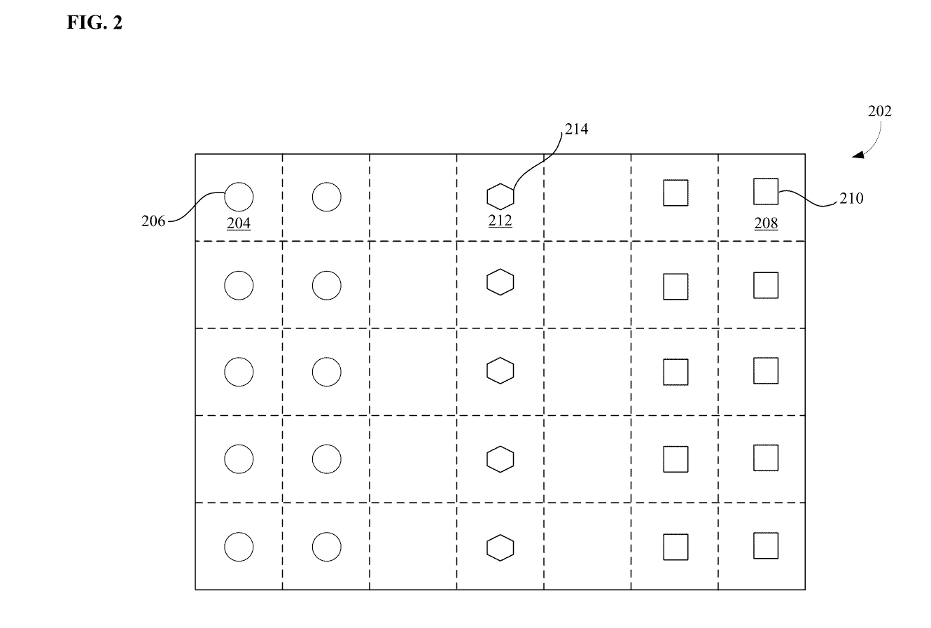

Referring to FIG. 2, in an embodiment, a unit dose package 202 includes a first storage location 204 having a first unit dose 206 of at least one phyto-cannabinoid and a pharmaceutically acceptable carrier. In an embodiment, a unit dose package 202 includes at least a second storage location 208 having a second unit dose 210 of at least one phyto-cannabinoid and a pharmaceutically acceptable carrier. In an embodiment, the second unit dose 210 is different from the first unit dose 206.

In an embodiment, the first unit dose 206 or the second unit dose 210 comprises from about 0.1 milligram to about 500 milligrams of at least one active agent. In an embodiment, the first unit dose 206 or the second unit dose 210 comprises from about 0.5 milligram to about 250 milligrams of at least one active agent. In an embodiment, the first unit dose 206 or the second unit dose 210 comprises from about 1 milligrams to about 100 milligrams of at least one active agent.

In an embodiment, the first unit dose 206 or the second unit dose 210 comprises at least about 0.1 milligram of at least one phyto-cannabinoid. In an embodiment, the first unit dose 206 or the second unit dose 210 comprises at least about 0.5 milligram of at least one phyto-cannabinoid. In an embodiment, the first unit dose 206 or the second unit dose 210 comprises at least about 1 milligram of at least one phyto-cannabinoid. In an embodiment, the first unit dose 206 or the second unit dose 210 comprises at least about 1.5 milligrams of at least one phyto-cannabinoid. In an embodiment, the first unit dose 206 or the second unit dose 210 comprises about 5 milligrams of at least one phyto-cannabinoid. In an embodiment, the first unit dose 206 or the second unit dose 210 comprises about 10 milligrams of the at least one phyto-cannabinoid. In an embodiment, the first unit dose 206 or the second unit dose 210 comprises about 15 milligrams of the at least one phyto-cannabinoid. In an embodiment, the first unit dose 206 or the second unit dose 210 comprises about 20 milligrams of the at least one phyto-cannabinoid. In an embodiment, the first unit dose 206 or the second unit dose 210 comprises about 25 milligrams of the at least one phyto-cannabinoid. In an embodiment, the first unit dose 206 or the second unit dose 210 comprises about 50 milligrams of the at least one phyto-cannabinoid. In an embodiment, the at least one phyto-cannabinoid is tetrahydrocannabinol or an analogue or derivative thereof. In an embodiment, the at least one phyto-cannabinoid is Δ⁹-tetrahydrocannabinol or an analogue or derivative thereof. In an embodiment, the at least one phyto-cannabinoid is Δ⁸-tetrahydrocannabinol or an analogue or derivative thereof.

In an embodiment, the second unit dose 210 comprises a geometric shape different from the geometric shape of the first unit dose 206. In an embodiment, the geometric shape is indicative of a dose amount. In an embodiment, the shape of the unit dose can be a geometrical shape including regular geometric shapes, such as circular, hexagonal, pentagonal, rectangular, triangular, and the like, as well as irregular geometric shapes. In an embodiment, the number of facets in the shape is indicative of a dose amount. For example, in an embodiment, each facet has a shape indicative of a dose amount increment (e.g., one facet for every 5 milligram of active agent). In an embodiment, the geometric shape is indicative of a delivery mode.

In an embodiment, the second unit dose 210 comprises a color different from the color of the first unit dose 206. In an embodiment, the color is indicative of a dose amount (e.g., red is indicative of 5 milligrams of active agent, green is indicative of 10 milligrams of active agent, blue is indicative of 15 milligrams of active agent, white is indicative of 25 milligrams of active agent, etc.). In an embodiment, the color is indicative of a delivery mode (e.g., red is indicative of inhaled, green is indicative of ingested, blue is indicative of sublingual, white is indicative of transdermal, beige is indicative of topical, purple is indicative of transmucosal, brow is indicative of rectal, etc.).

In an embodiment, the first unit dose 206 comprises one of an inhalable unit dose, an ingestible unit dose, a sublingual unit dose, a transdermal unit dose, a topical unit dose, a transmucosal unit dose, or a rectal unit dose; and the second unit dose 210 comprises a different one of an inhalable unit dose, an ingestible unit dose, a sublingual unit dose, a transdermal unit dose, a topical unit dose, a transmucosal unit dose, or a rectal unit dose.

In an embodiment, the first unit dose 206 comprises at least one terpene (e.g., borneol, β-caryophyllene, cineole, delta-3-carene, limonene, D-linalool, β-myrcene, pinene, pulegone, sabinene, terpineol, and the like). In an embodiment, the first unit dose 206 comprises at least a first terpene. In an embodiment, the first unit dose 206 comprises a second terpene. In an embodiment, the first unit dose 206 comprises a third terpene. In an embodiment, the first unit dose 206 comprises a fourth terpene. In an embodiment, the second unit dose 210 comprises at least one terpene. In an embodiment, the second unit dose 210 comprises a second terpene. In an embodiment, the second unit dose 210 comprises a third terpene. In an embodiment, the second unit dose 210 comprises a fourth terpene.

In an embodiment, the second unit dose 210 comprises a terpene different from a terpene of the first unit dose 206. In an embodiment, the second unit dose 210 comprises a terpene amount different from a terpene amount of the first unit dose 206. In an embodiment, the second unit dose 210 comprises a terpene composition mixture different from a terpene composition mixture of the first unit dose 206. In an embodiment, the first unit dose 206 comprises one of borneol, 1-caryophyllene, cineole, delta-3-carene, limonene, D-linalool, β-myrcene, pinene, pulegone, sabinene, or terpineol, and the second unit dose 210 comprises a different one of borneol, β-caryophyllene, cineole, delta-3-carene, limonene, D-linalool, β-myrcene, pinene, pulegone, sabinene, or terpineol. In an embodiment, first unit dose 206 comprises a mixture of two or more of borneol, β-caryophyllene, cineole, delta-3-carene, limonene, D-linalool, β-myrcene, pinene, pulegone, sabinene, or terpineol, and the second unit dose 210 comprises a different mixture of two or more of borneol, β-caryophyllene, cineole, delta-3-carene, limonene, D-linalool, β-myrcene, pinene, pulegone, sabinene, or terpineol.

In an embodiment, the second unit dose 210 comprises a phyto-cannabinoid amount different from the first unit dose 206. In an embodiment, the second unit dose 210 comprises a phyto-cannabinoid ratio different from the phyto-cannabinoid ratio of the first unit dose 206. In an embodiment, the second unit dose 210 comprises a phyto-cannabinoid:terpene ratio different from a phyto-cannabinoid:terpene ratio of the first unit dose 206. In an embodiment, the second unit dose 210 comprises a phyto-cannabinoid:terpene:phyto-cannabidiol ratio different from a phyto-cannabinoid:terpene:phyto-cannabidiol ratio of the first unit dose 206.

In an embodiment, the first unit dose 206 or the second unit dose 210 comprises a pellet. In an embodiment, the first unit dose 206 or the second unit dose 210 comprises a plurality of pellets. In an embodiment, the first unit dose 206 or the second unit dose 210 comprises a substantially solid dose form. In an embodiment, the first unit dose 206 or the second unit dose 210 comprises a tablet.

In an embodiment, the first unit dose 206 or the second unit dose 210 comprises one or more of a phyto-cannabichromene (CBC), a phyto-cannabidiol (CBD), a phyto-cannabidiolic Acid (CBD-A), a phyto-cannabigerol (CBG), a phyto-cannabinol (CBN), a cannabidivarin (CBDV), a phyto-tetrahydrocannabinolic acid (THC-A), and a phyto-tetrahydrocannabivarin (THCV). In an embodiment, the first unit dose 206 comprises one of a phyto-cannabichromene (CBC), a phyto-cannabidiol (CBD), a phyto-cannabidiolic Acid (CBD-A), a phyto-cannabigerol (CBG), a phyto-cannabinol (CBN), a cannabidivarin (CBDV), a phyto-tetrahydrocannabinolic acid (THC-A), and a phyto-tetrahydrocannabivarin (THCV), and the second unit dose 210 comprises a different one of a phyto-cannabichromene (CBC), a phyto-cannabidiol (CBD), a phyto-cannabidiolic Acid (CBD-A), a phyto-cannabigerol (CBG), a phyto-cannabinol (CBN), a cannabidivarin (CBDV), a phyto-tetrahydrocannabinolic acid (THC-A), and a phyto-tetrahydrocannabivarin (THCV).

In an embodiment, the pharmaceutically acceptable carrier of the first unit dose 206 comprises a solid, a liquid, a solution, a suspension, a gel, a glass, a solid dispersion, an ointment, or a lotion; and the pharmaceutically acceptable carrier of the second unit dose 210 comprises a different one of a solid, a liquid, a solution, a suspension, a gel, a glass, a solid dispersion, an ointment, or a lotion.

In an embodiment, the first unit dose 206 comprises one of an inhalable pharmaceutically acceptable carrier, an ingestible pharmaceutically acceptable carrier, a sublingual pharmaceutically acceptable carrier, a transdermal pharmaceutically acceptable carrier, a topical pharmaceutically acceptable carrier, a transmucosal pharmaceutically acceptable carrier, or a rectal pharmaceutically acceptable carrier; and the second unit dose 210 comprises a different one of an inhalable pharmaceutically acceptable carrier, an ingestible pharmaceutically acceptable carrier, a sublingual pharmaceutically acceptable carrier, a transdermal pharmaceutically acceptable carrier, a topical pharmaceutically acceptable carrier, a transmucosal pharmaceutically acceptable carrier, or a rectal pharmaceutically acceptable carrier.

In an embodiment, a sublingual unit dose form includes at least one phyto-cannabinoid and a pharmaceutically acceptable carrier. In an embodiment, the at least one phyto-cannabinoid is a tetrahydrocannabinol or an analogue or derivative thereof. In an embodiment, the at least one phyto-cannabinoid is Δ⁹-tetrahydrocannabinol or an analogue or derivative thereof. In an embodiment, the at least one phyto-cannabinoid is Δ⁸-tetrahydrocannabinol or an analogue or derivative thereof.

In an embodiment, a sublingual unit dose form includes at least one phyto-cannabinoid, at least one terpene, and a pharmaceutically acceptable carrier. Non-limiting examples of terpenes include borneol, β-caryophyllene, cineole, delta-3-carene, limonene, D-linalool, β-myrcene, pinene, pulegone, sabinene, terpineol, and the like. In an embodiment, a sublingual unit dose form includes at least one phyto-cannabinoid and one or more phyto-cannabichromenes (CBC), phyto-cannabidiols (CBD), phyto-cannabidiolic acids (CBD-A), phyto-cannabigerols (CBG), phyto-cannabinols (CBN), phyto-cannabidivarins (CBDV), phyto-tetrahydrocannabinolic acids (THC-A), or phyto-tetrahydrocannabivarins (THCV).

In an embodiment, a sublingual unit dose form includes at least one phyto-cannabinoid and at least one nonselective COX inhibitor. In an embodiment, a sublingual unit dose form includes at least one phyto-cannabinoid, at least one nonselective COX inhibitor, and at least one monoamine oxidase inhibitor. In an embodiment, a sublingual unit dose form includes at least one phyto-cannabinoid, at least one nonselective COX inhibitor, and at least one nonselective monoamine oxidase inhibitor. In an embodiment, a sublingual unit dose form includes at least one phyto-cannabinoid and at least one nootropic agent.

In an embodiment, the substantially solid sublingual unit dose form comprises a solid dispersion. In an embodiment, the sublingual unit dose form comprises a pellet. In an embodiment, the sublingual unit dose form comprises a plurality of pellets. In an embodiment, the sublingual unit dose form comprises a tablet.

In an embodiment, the pharmaceutically acceptable carrier comprises a polymeric carrier. In an embodiment, the pharmaceutically acceptable carrier comprises a solid, a liquid, a solution, a suspension, a gel, a glass, a solid dispersion, or combinations thereof. In an embodiment, the pharmaceutically acceptable carrier comprises one or more of oils, glycerides, triglycerides, chitosan, cyclodextrins, pullulan, and the like. In an embodiment, the pharmaceutically acceptable carrier comprises a plurality of carrier particles at least partially covered by particles including the at least one phyto-cannabinoid.

In an embodiment, the unit dose package 202 includes at least a third storage location 212 having a unit dose of a nonselective COX inhibitor 214 (e.g., aspirin, ibuprofen, naproxen, and the like). In an embodiment, the unit dose package 202 includes at least a third storage location 212 having a unit dose 214, such as a unit dose of a nonselective COX inhibitor and a monoamine oxidase inhibitor. Non-limiting examples of monoamine oxidase inhibitors include anthocyanins, curcumin, ginkgo biloba, proanthocyanidin, rhodiola rosea, ruta graveolens, and the like. In an embodiment, the unit dose package 202 includes at least a third storage location 212 having a unit dose of a nonselective COX inhibitor and a nonselective monoamine oxidase inhibitor.

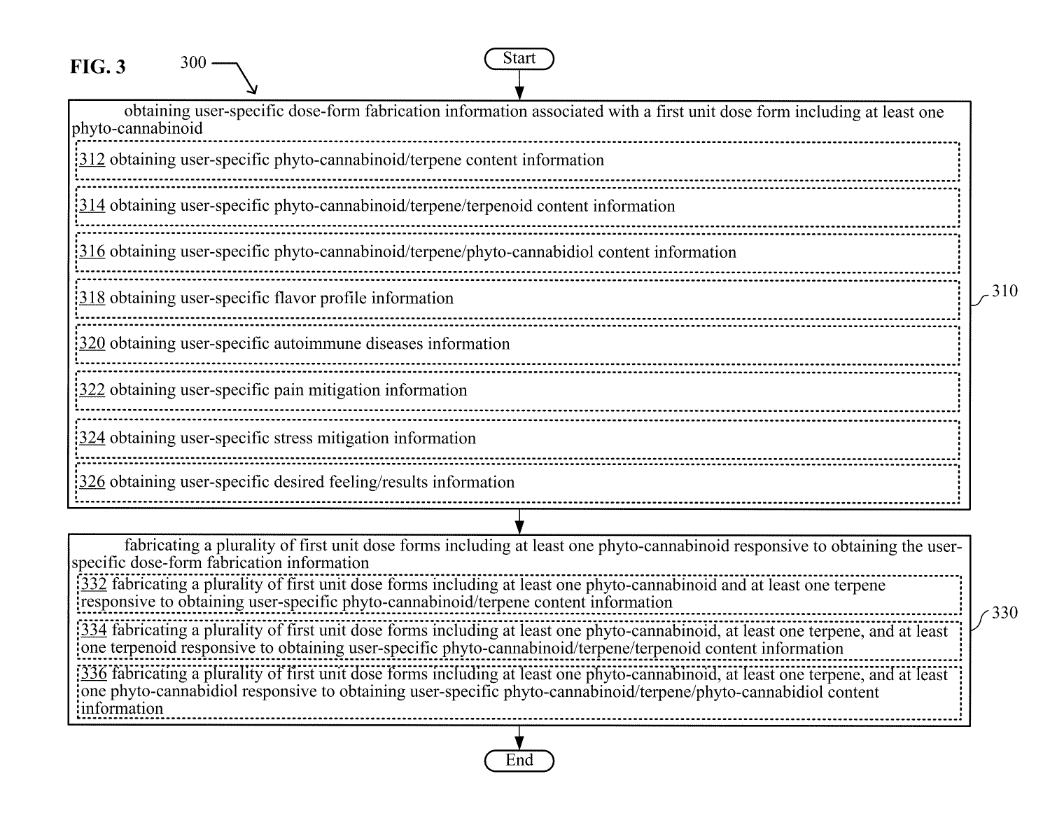

FIG. 3 shows a method 300. At 310, the method 300 includes obtaining user-specific dose-form fabrication information associated with a first unit dose 106 form including at least one phyto-cannabinoid. At 312, obtaining the user-specific dose-form fabrication information associated with a first unit dose 106 form including at least one phyto-cannabinoid includes obtaining user-specific phyto-cannabinoid/terpene content information. At 314, obtaining the user-specific dose-form fabrication information associated with a first unit dose 106 form including at least one phyto-cannabinoid includes obtaining user-specific phyto-cannabinoid/terpene/terpenoid content information. At 316, obtaining the user-specific dose-form fabrication information associated with a first unit dose 106 form including at least one phyto-cannabinoid includes obtaining user-specific phyto-cannabinoid/terpene/phyto-cannabidiol content information. At 318, obtaining the user-specific dose-form fabrication information associated with a first unit dose 106 form including at least one phyto-cannabinoid includes obtaining user-specific flavor profile information.

At 320, obtaining the user-specific dose-form fabrication information associated with a first unit dose 106 form including at least one phyto-cannabinoid includes obtaining user-specific autoimmune diseases information. At 322, obtaining the user-specific dose-form fabrication information associated with a first unit dose 106 form including at least one phyto-cannabinoid includes obtaining user-specific pain mitigation information. At 324, obtaining the user-specific dose-form fabrication information associated with a first unit dose 106 form including at least one phyto-cannabinoid includes obtaining user-specific stress mitigation information. At 326, obtaining the user-specific dose-form fabrication information associated with a first unit dose 106 form including at least one phyto-cannabinoid includes obtaining user-specific desired feeling/results information.

At 330, the method 300 includes fabricating a plurality of first unit dose 106 forms including at least one phyto-cannabinoid responsive to obtaining the user-specific dose-form fabrication information. At 332, fabricating the plurality of first unit dose 106 forms including at least one phyto-cannabinoid includes fabricating a plurality of first unit dose 106 forms including at least one phyto-cannabinoid and at least one terpene responsive to obtaining user-specific phyto-cannabinoid/terpene content information. At 334, fabricating the plurality of first unit dose 106 forms including at least one phyto-cannabinoid includes fabricating a plurality of first unit dose 106 forms including at least one phyto-cannabinoid, at least one terpene, and at least one terpenoid responsive to obtaining user-specific phyto-cannabinoid/terpene/terpenoid content information. At 336, fabricating the plurality of first unit dose 106 forms including at least one phyto-cannabinoid includes fabricating a plurality of first unit dose 106 forms including at least one phyto-cannabinoid, at least one terpene, and at least one phyto-cannabidiol responsive to obtaining user-specific phyto-cannabinoid/terpene/phyto-cannabidiol content information.

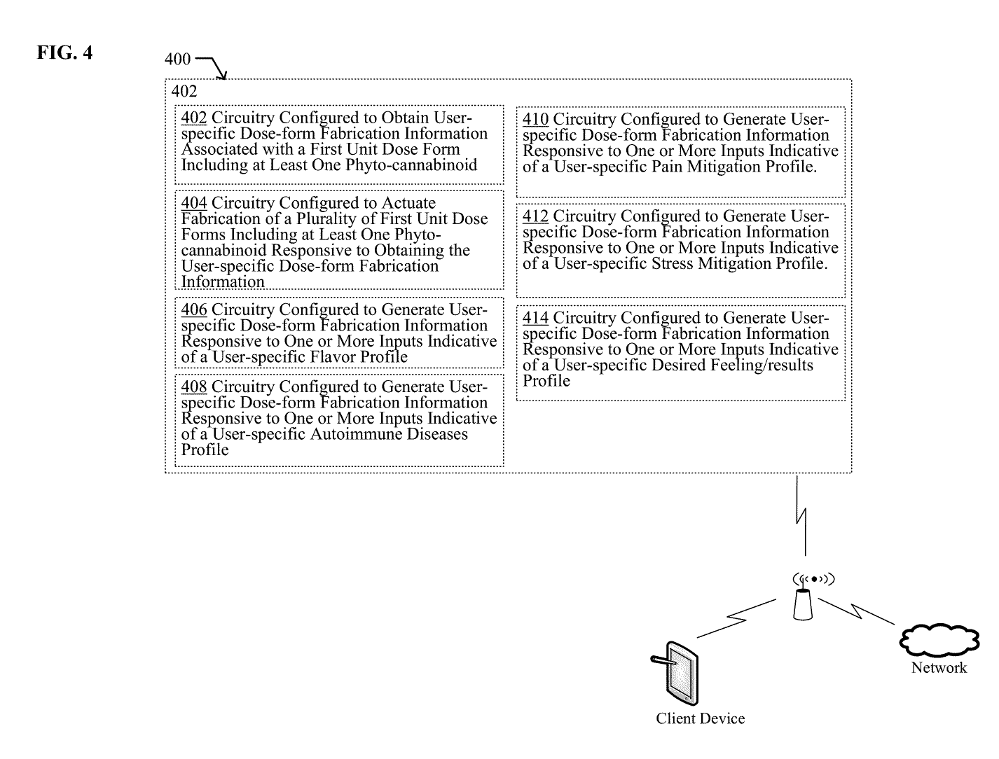

Referring to FIG. 4, in an embodiment, a system 400 includes circuitry 402 configured to obtain user-specific dose-form fabrication information associated with a first unit dose 106 form including at least one phyto-cannabinoid. In an embodiment, a system 400 includes circuitry 404 configured to actuate fabrication of a plurality of first unit dose 106 forms including at least one phyto-cannabinoid responsive to obtaining the user-specific dose-form fabrication information. In an embodiment, a system 400 includes circuitry 406 configured to generate user-specific dose-form fabrication information responsive to one or more inputs indicative of a user-specific flavor profile. In an embodiment, a system 400 includes circuitry 408 configured to generate user-specific dose-form fabrication information responsive to one or more inputs indicative of a user-specific autoimmune diseases profile.

In an embodiment, a system 400 includes circuitry 410 configured to generate user-specific dose-form fabrication information responsive to one or more inputs indicative of a user-specific pain mitigation profile. In an embodiment, a system 400 includes circuitry 412 configured to generate user-specific dose-form fabrication information responsive to one or more inputs indicative of a user-specific stress mitigation profile. In an embodiment, a system 400 includes circuitry 414 configured to generate user-specific dose-form fabrication information responsive to one or more inputs indicative of a user-specific desired feeling/results profile.

U.S. Application Nos. 62/027,391 and 62/027,374 are incorporated herein by reference in their entireties. In addition, the embodiments, features, systems, devices, materials, methods, and techniques described herein may, in certain embodiments, be applied to or used in connection with any one or more of the embodiments, features, systems, devices, compositions, materials, methods, and techniques disclosed in the above-mentioned U.S. Application Nos. 62/027,391 and 62/027,374. At least some embodiments disclosed herein can be packaging for containing doses of medication. The doses can be individual solid or liquid medication units that can be dispensed over a period of time. In some embodiments, the packaging includes sealed compartments for holding the same or different units of medication. A user can selectively open compartments to access each dose. For example, a unit dose package can be a blister pack with a backing member and a blister mounted defining individual cavities for storing doses medication. Each cavity can contain a single dose, such as a pill. In some embodiments, the packaging includes single or multi-dose unit dose packages in the form of packets, pouches, or the like. Other types of packaging can be used and can be include closures, lids, break-away member, cavities, or combinations thereof. These packages can be reusable (e.g., reclosable) or single use (e.g., non-reclosable). Unit dose packaging may include any desired number of doses and may include tamper evidence, child-resistance features and may include labeling with, for example, dose information, expiration dates, machine readable information (e.g., barcodes), or other information.

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and/or “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). Typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the operations recited therein generally may be performed in any order. Also, although various operational flows are presented in a sequence(s), it should be understood that the various operations may be performed in orders other than those that are illustrated, or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1. A unit dose package, comprising:
at least a first storage location having a first unit dose of at least one phyto-cannabinoid; and
a pharmaceutically acceptable carrier.

2. The unit dose package of claim 1, wherein the first unit dose comprises from about 0.1 milligrams to about 500 milligrams of the at least one phyto-cannabinoid.

3-8. (canceled)

9. The unit dose package of claim 1, wherein the first unit dose comprises a first terpene.

10-15. (canceled)

16. The unit dose package of claim 1, wherein the pharmaceutically acceptable carrier comprises a controlled-release carrier.

17. (canceled)

18. (canceled)

19. (canceled)

20. The unit dose package of claim 1, wherein the pharmaceutically acceptable carrier includes a disintegrant in an amount sufficient to cause the at least one phyto-cannabinoid to exhibit an immediate release profile.

21. The unit dose package of claim 1, wherein the first unit dose comprises a geometric shape indicative of a dose amount.

22. The unit dose package of claim 1, wherein the first unit dose comprises a color indicative of a dose amount.

23. (canceled)

24. (canceled)

25. The unit dose package of claim 1, comprising:
at least a second storage location having a unit dose of a nonselective COX inhibitor.

26. (canceled)

27. (canceled)

28. The unit dose package of claim 1, wherein the unit dose package comprise a communication component that informs a network application regarding contents.

29. (canceled)

30. The unit dose package of claim 1, wherein the unit dose package comprise circuitry configured to initiate a discovery protocol that allows the unit dose package and a client device to identify each other and negotiate one or more pre-shared keys.

31. (canceled)

32. A unit dose package, comprising:
a first storage location having a first unit dose of at least one phyto-cannabinoid and a pharmaceutically acceptable carrier; and
at least a second storage location having a second unit dose of at least one phyto-cannabinoid and a pharmaceutically acceptable carrier, the second unit dose different from the first unit dose.

33-38. (canceled)

39. The unit dose package of claim 32, wherein the second unit dose comprises a phyto-cannabinoid amount different from the first unit dose.

40. (canceled)

41. (canceled)

42. The unit dose package of claim 32, wherein the first unit dose comprises one of an inhalable unit dose, an ingestible unit dose, a sublingual unit dose, a transdermal unit dose, a topical unit dose, a transmucosal unit dose, or rectal unit dose; and the second unit dose comprises a different one of an inhalable unit dose, an ingestible unit dose, a sublingual unit dose, a transdermal unit dose, a topical unit dose, a transmucosal unit dose, or rectal unit dose.

43. The unit dose package of claim 32, wherein the first unit dose comprises at least one terpene.

44. (canceled)

45. (canceled)

46. (canceled)

47. The unit dose package of claim 32, wherein the second unit dose comprises a phyto-cannabinoid:terpene ratio different from a phyto-cannabinoid:terpene ratio of the first unit dose.

48. (canceled)

49. The unit dose package of claim 32, wherein the second unit dose comprises a terpene composition mixture different from a terpene composition mixture of the first unit dose.

50-59. (canceled)

60. The unit dose package of claim 32, wherein the first unit dose comprises one of a phyto-cannabichromene, a phyto-cannabidiol, a phyto-cannabidiolic acid, a phyto-cannabigerol, a phyto-cannabinol, a phyto-cannabidivarin, a phyto-tetrahydrocannabinolic acid, and a phyto-tetrahydrocannabivarin, and the second unit dose comprises a different one of a phyto-cannabichromene, a phyto-cannabidiol, a phyto-cannabidiolic acid, a phyto-cannabigerol, a phyto-cannabinol, a phyto-cannabidivarin, a phyto-tetrahydrocannabinolic acid, and a phyto-tetrahydrocannabivarin.

61. (canceled)

62. (canceled)

63. The unit dose package of claim 32, wherein the pharmaceutically acceptable carrier of the first unit dose comprises a solid, a liquid, a solution, a suspension, a gel, a glass, a solid dispersion, an ointment, or a lotion; and the pharmaceutically acceptable carrier of the second unit dose comprises a different one of a solid, a liquid, a solution, a suspension, a gel, a glass, a solid dispersion, an ointment, or a lotion.

64. A sublingual unit dose form, comprising:
at least one phyto-cannabinoid; and
a pharmaceutically acceptable carrier.

65. (canceled)

66. The sublingual unit dose form of claim 64, wherein the sublingual unit dose form comprises a plurality of pellets.

67. (canceled)

68. The sublingual unit dose form of claim 64, wherein the substantially solid sublingual unit dose comprises a solid dispersion.

69. (canceled)

70. The sublingual unit dose form of claim 64, wherein the pharmaceutically acceptable carrier comprises a plurality of carrier particles at least partially covered by particles including the at least one phyto-cannabinoid.

71. (canceled)

72. (canceled)

73. (canceled)

74. (canceled)

75. A method, comprising:
obtaining user-specific dose-form fabrication information associated with a first unit dose form including at least one phyto-cannabinoid; and
fabricating a plurality of first unit dose forms including at least one phyto-cannabinoid responsive to obtaining the user-specific dose-form fabrication information.

76. (canceled)

77. (canceled)

78. (canceled)

79. The method of claim 75, wherein obtaining the user-specific dose-form fabrication information associated with a first unit dose form including at least one phyto-cannabinoid includes obtaining user-specific flavor profile information

80. The method of claim 75, wherein obtaining the user-specific dose-form fabrication information associated with a first unit dose form including at least one phyto-cannabinoid includes obtaining user-specific auto-immune diseases information.

81. (canceled)

82. The method of claim 75, wherein obtaining the user-specific dose-form fabrication information associated with a first unit dose form including at least one phyto-cannabinoid includes obtaining user-specific stress mitigation information.

83. (canceled)

84. The method of claim 75, wherein fabricating the plurality of first unit dose forms including at least one phyto-cannabinoid includes fabricating a plurality of first unit dose forms including at least one phyto-cannabinoid and at least one terpene responsive to obtaining user-specific phyto-cannabinoid/terpene content information.

85. (canceled)

86. (canceled)

87. A system, comprising:
circuitry configured to obtain user-specific dose-form fabrication information associated with a first unit dose form including at least one phyto-cannabinoid; and
circuitry configured to actuate fabrication of a plurality of first unit dose forms including at least one phyto-cannabinoid responsive to obtaining the user-specific dose-form fabrication information.

88. The system of claim 87, comprising:
circuitry configured to generate user-specific dose-form fabrication information responsive to one or more inputs indicative of a user-specific flavor profile.

89. The system of claim 87, comprising:
circuitry configured to generate user-specific dose-form fabrication information responsive to one or more inputs indicative of a user-specific auto-immune diseases profile.

90. The system of claim 87, comprising:
circuitry configured to generate user-specific dose-form fabrication information responsive to one or more inputs indicative of a user-specific pain mitigation profile.

91. The system of claim 87, comprising:
circuitry configured to generate user-specific dose-form fabrication information responsive to one or more inputs indicative of a user-specific stress mitigation profile.

92. The system of claim 87, comprising:
circuitry configured to generate user-specific dose-form fabrication information responsive to one or more inputs indicative of a user-specific desired feeling/results profile.
